(19) United States
(12) Patent Application Publication
(10) Pub. No.: US 2020/0171222 A1
(43) Pub. Date: Jun. 4, 2020

(54) CHEST TUBE MEMBRANE

(71) Applicants: Chris Salvino, SCOTTSDALE, AZ (US); Keir Hart, Lafayette, CO (US); Mark J. Huseman, Broomfield, CO (US)

(72) Inventors: Chris Salvino, SCOTTSDALE, AZ (US); Keir Hart, Lafayette, CO (US); Mark J. Huseman, Broomfield, CO (US)

(21) Appl. No.: 16/699,887

(22) Filed: Dec. 2, 2019

Related U.S. Application Data
(60) Provisional application No. 62/773,754, filed on Nov. 30, 2018.

(51) Int. Cl. A61M 1/04; A61M 39/02

(52) U.S. Cl. CPC A61M 1/04; A61M 2039/027; A61M 39/0247

(57) ABSTRACT

An integrated chest evacuation tube that is specifically configured to function as a closed tube when inserted and retained inside of a chest cavity to provide enhanced benefits over the state of the art. The integrated chest evacuation tube generally includes a fluid impermeable membrane fixedly attached to a single flexible hollow tube. The fluid impermeable membrane is interposed between an inlet port and an outlet port or optionally at said outlet port of a chest tube. The membrane is configured to seal the tube's passageway from any transfer of the fluid. The impermeable membrane is adapted to be ruptured by a force other than pressure from said fluid.

### CROSS-REFERENCE TO RELATED APPLICATIONS

This application claims priority to and the benefit of U.S. Provisional Patent Application No. 62/773,754 entitled: Chest Tube Membrane, filed on Nov. 30, 2018.

### FIELD OF THE INVENTION

The present embodiments are directed to an integrated chest evacuation tube and membrane system with some applications being used with a chest tube insertion device.

### DESCRIPTION OF RELATED ART

The lungs are surrounded by a pleural sac made up of two membranes, the visceral and parietal pleurae. The parietal pleura lines the thoracic wall, and the visceral pleura surrounds the lung. The pleural space is a potential space between these two layers of pleurae. It contains a thin layer of serous pleural fluid that provides lubrication for the pleurae and allows the layers of pleurae to smoothly slide over each other during respiration. In abnormal circumstances, the pleural space can fill with air and certain types of fluids not normally present requiring drainage.

In the industrialized world, trauma is the leading cause of death in males under the age of forty. In the United States, chest injuries are responsible for one-fourth of all trauma deaths. Many of these fatalities could be prevented by early recognition of the injury followed by prompt management. Some traumatic chest injuries require quick placement of chest tubes to drain out air and/or fluids (such as blood) from the chest cavity.

Several techniques are currently used to insert a chest tube, each of which involves a relatively lengthy manual procedure that requires knowledge and experience. The most common technique involves surgical preparation and draping at the site of the tube insertion (usually at the nipple level-fifth intercostal space, anterior to the mid-axillary line on the affected side), administering local anesthesia to the insertion site, and making a 2-4 cm vertical incision. A clamp is inserted through the incision and spread, tearing muscle and tissue until a tract large enough to accept a finger is created. Next, the parietal pleura is punctured. One way to puncture the parietal pleura is with the tip of a clamp, and the physician, on occasion, places a gloved finger into the incision to confirm the presence of a (locally) free pleural space. Next, the proximal end of the chest tube 145 (FIG. 1) is advanced through the incision into the pleural space. As the chest tube is inserted, it is sometimes directed posteriorly and superiorly towards the apex of the lung or elsewhere in the chest cavity. The goal is for the chest tube to drain the pleural space of both air and/or fluids such as blood. Accordingly, once the chest tube is appropriately in place to clear air and/or fluids (such as blood, infection, a transudate) from the pleural space, the tube is fixed to the skin with sutures around the tube anchoring the tube to the skin, dressing is applied, and the tube covered with a sterile dressing.

Inserting a chest tube using this standard technique can require more than 15 minutes to accomplish by a physician, requires extensive medical training to be performed properly and can be extremely painful as it is a difficult area to anesthetize due to the intercostal nerve that runs on the bottom of every rib. Further, while performing the procedure, the physician must attend to the patient receiving the chest tube and thus is precluded from attending to other patients.

FIG. 1A depicts a prior art chest tube insertion gun 100 which functions as a chest tube deployment device described in U.S. Pat. No. 7,811,293. This chest tube insertion gun 100 includes a housing 105, a handle 110 with the trigger 125, a probe tip 130 having a circular cutting tip 135 at the distal end thereof, a circular cross-sectioned cannula 140, and a circular cross sectioned chest tube 145. The circular cutting tip 135 rotates outside of the distal end up to a 90° angle of rotation (rotation angle) from its neutral position before rotating back to its neutral position. The circular cutting tip 135 is also able to rotate a small negative angle from its neutral position in order to retract inside of the distal end of the probe tip 130. The rotation angle works well for the circular cross-sectioned cannula 140.

FIG. 2A illustratively depicts a prior art side view drawing of different chest tube deployment device referred to as an actuator scalpel. Similar to the chest tube insertion gun 100, the actuator scalpel 200 provides a different handle system and the scalpel blade 220 that both rotates and travels outside of the probe tip 208 in a circular path. More specifically, the actuator scalpel comprises a handle body 202, a trigger 204, a probe 206, and a probe tip 208 showing the probe tip housing 212. The trigger 204 depicts finger grips 210 adapted to accommodate the fingers of a human hand (not shown). Shown for reference is the probe housing 211 and the body housing 205. In operation, the actuator scalpel 200 is gripped by an operator's (person's) palm positioned along the top of the handle body 207 with two of their fingers positioned in the finger grips 210 whereby upon squeezing the handle 204 towards the handle body 202, the scalpel 220 is made to move in a cutting motion.

FIG. 2B illustratively depicts a top view of the actuator scalpel 200 next to a prior art cannula 140. The cannula 140 is a linear tube (or in some cases arc-shaped, not shown) that is arranged to slide over the probe tip 208 and cover the probe shaft 206 via a base opening 102 and a distal end opening 104. In practice, with the cannula 140 slid over the probe shaft 206, which essentially covers the probe 206, the actuator scalpel 200 is made to cut a pathway into the chest cavity of the patient whereby the cannula 140 is slid off of the probe tip 208 and thereby deployed into the chest of a patient. Accordingly, the probe 206 serves as a chest tube deployment shaft. The cannula 140 provides a drainage pathway for fluid to escape the patient.

FIG. 3A-3C illustratively depict drawings of a prior art Heimlich valve in different states of operation used with a chest tube. More specifically, FIG. 3A shows a side view of a Heimlich valve 270 in an inactive, or unused, state/configuration. As viewed in conjunction with FIG. 3D, the Heimlich valve 270 has an inlet nozzle 276 that is adapted to press into the end of a pliable hollow tube 282 whereby the actual Heimlich valve 270 is always outside of a tube, which could be a chest tube (not shown), a pliable rubber sleeve 272, and an outlet nozzle 274 adapted to press into the end of a pliable hollow outlet tube 278 that leads to a fluid collection bag 280. FIG. 3B depicts the pliable rubber sleeve 272 closed, i.e., sealed off when air and/or fluid flows into the outlet nozzle 274 thereby preventing air and/or fluid flowing into a human's chest cavity 288. FIG. 3C depicts the pliable rubber sleeve 272 opened to allow air and/or fluid to flow in the proper direction through the inlet nozzle 276, through the Heimlich Valve body, and out the outlet nozzle 274.

FIG. 3D illustratively depicts a drawing of a prior art drainage system with the Heimlich Valve assembly engaged with a human patient. More specifically, a chest tube 145 is inserted into a lung space 286 via an incision 290 in a chest cavity 288 of a human patient/subject 299. The chest tube 145 is connected to a valve 284 that allows an operator (not shown) to open and close passage between the chest tube 145 and the fluid collection bag 280. Connected to the other side of the pigtail valve 284 is an intermediate pliable hollow tube 282 pressed into the inlet nozzle 276 of the Heimlich valve 270. The outlet nozzle 274 of the Heimlich valve 270 is pressed into a pliable hollow outlet tube 278 which is connected to the fluid collection bag 280. Accordingly, there is a direct path between the lung space 286 and the fluid collection bag 280. As can be readily appreciated, fluid and/or air from the fluid collection bag 280 is prevented from back flowing into the subject's/patient 299. Though there are numerous advantages to the present a state-of-the-art Heimlich Valve 270 and the assembly shown in FIG. 3D, applications in an emergency situation can be cumbersome and problematic.

It is to innovations related to this subject matter that the claimed invention is generally directed.

### SUMMARY OF THE INVENTION

The present embodiments are directed to essentially an impermeable membrane that blocks fluid from exiting a chest tube upon deployment in a chest. The impermeable membrane can be ruptured to facilitate movement of the fluid through the chest tube when it is convenient to do so.

Certain embodiments of the present invention contemplate a single use chest evacuation tube consisting: an inlet port adapted to intake fluid from a human chest cavity into said single use chest evacuation tube; an outlet port adapted to expel said fluid out there through; a single flexible hollow tube defining a passageway between said inlet port and said outlet port, said single flexible hollow tube adapted to transport said fluid from said input port to said output port; and a fluid impermeable membrane 400 fixedly attached to said single flexible hollow tube, said fluid impermeable membrane 400 interposed between said inlet port 408 and said outlet port 404 or at said outlet port 404, said membrane 400 configured to seal said passageway 420 from said fluid exiting said outlet port 404, said impermeable membrane 400 configured to be ruptured by a force other than pressure from said fluid.

Yet other certain embodiments of the present invention contemplate a method comprising: providing a single use chest evacuation tube, an outlet port, a single passageway between said inlet port and said outlet port, a membrane fixedly attached to said single use chest evacuation tube, said membrane fully blocks said passageway; pushing the inlet port into a human chest cavity under pressure with chest fluid; after said pushing step, unblocking said passageway by rupturing said membrane through physical manipulation that does not include pressure from said chest fluid; and after said rupturing step, directing said chest fluid that enters said inlet port, passes through said passageway, passes through said ruptured membrane and out though said outlet port.

While other certain embodiments of the present invention contemplate a closed system chest evacuation tube comprising: an inlet port adapted to intake fluid from a human chest cavity into said single use chest evacuation tube; an outlet port adapted to expel said fluid out of said single use chest evacuation tube through said outlet port; one passageway between said inlet port and said outlet port, said passageway configured to transport said fluid from said input port to said output port; and a membrane that is impermeable to said fluid, said membrane fixedly attached to said single use chest evacuation tube, said membrane fully blocks said passageway, said membrane configured to rupture by though physical manipulation other than pressure from said fluid.

### BRIEF DESCRIPTION OF THE DRAWINGS

FIG. 1 illustratively depicts a prior art drawing chest tube insertion gun;

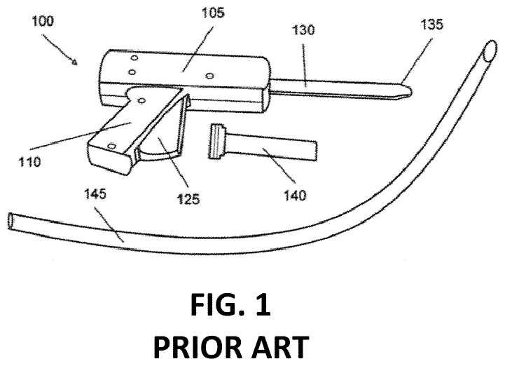

FIG. 2A illustratively depicts a front isometric view drawing of the prior art actuator scalpel of FIG. 2A;

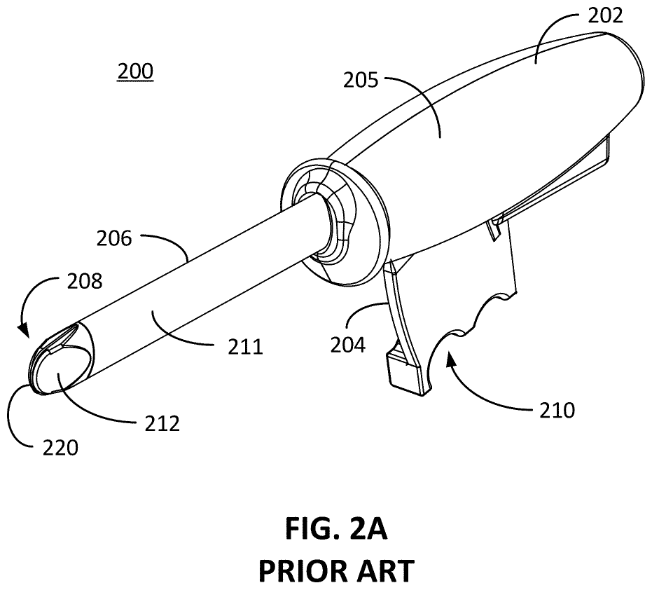

FIG. 2B illustratively depicts a drawing of a top view of the prior art actuator scalpel of FIG. 2A next to a prior art cannula;

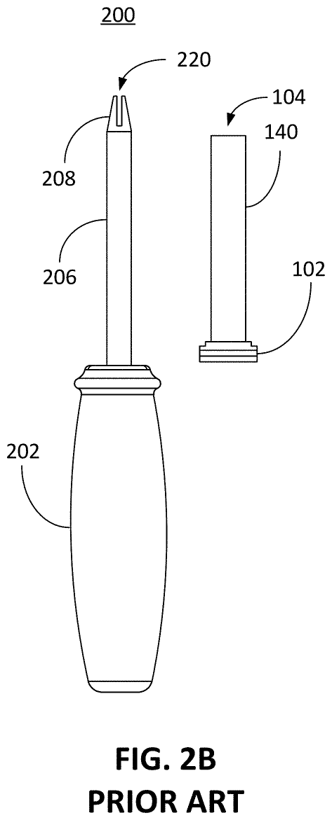

FIG. 3A-3C illustratively depict drawings of a prior art Heimlich Valve in different states of operation used with a chest tube;

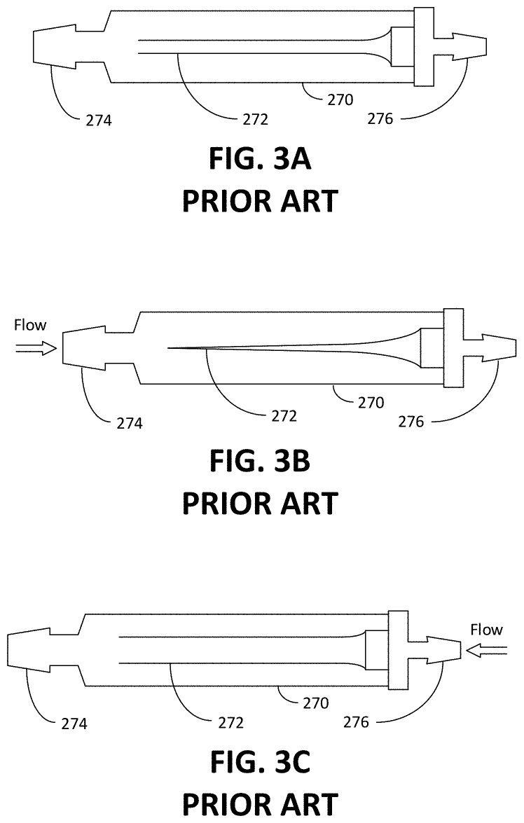

FIG. 3D illustratively depicts a drawing of a prior art close drainage system with the pigtail Heimlich Valve assembly engaged with a human patient;

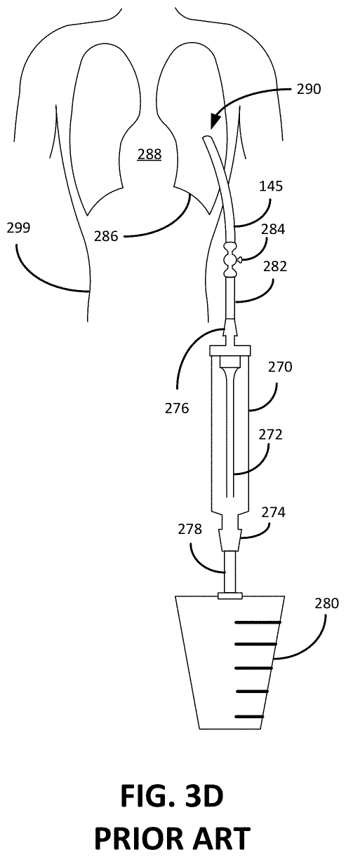

FIG. 4 illustratively depicts a perspective drawing of a single use chest evacuation tube system with embodiments of the present invention;

FIGS. 5A-5C illustratively depict drawings of a rupturable membrane embodiment consistent with embodiments of the present invention;

FIG. 6A-6C illustratively depicts line drawings of another membrane rupturing embodiment consistent with embodiments of the present invention;

FIG. 7 illustratively depicts a line drawing of another embodiment of a hinged bladder system consistent with embodiments of the present invention;

FIGS. 8A and 8B illustratively depict line drawings of yet another single use membrane-chest tube system consistent with embodiments of the present invention;

FIG. 9A illustratively depicts a typical location where an embodiment of the integrated chest evacuation tube and membrane system can be deployed; and

FIG. 9B illustratively depicts one embodiment of the chest evacuation tube integrated with a membrane embodiment being inserted between ribs of a patient/subject consistent with embodiments of the present invention.

### DETAILED DESCRIPTION

Initially, this disclosure is by way of example only, not by limitation. Thus, although the instrumentalities described herein are for the convenience of explanation, shown and described with respect to exemplary embodiments, it will be appreciated that the principles herein may be applied equally in other types of situations involving similar uses of an integrated chest evacuation tube and valve system. In what follows, similar or identical structures may (and may not) be identified using identical callouts.

Described herein are embodiments of an integrated chest evacuation tube that is specifically configured as a closed system when inserted and retained inside of a chest cavity. The disclosed closed system chest tube embodiments provide enhanced benefits over the state of the art by safeguarding medical personnel from fluid and/or air that may unintentionally escape through the chest tube when first inserted in a patient. Certain embodiments envision a chest evacuation tube generally including a fluid impermeable membrane blocking the pathway in a flexible chest tube. The fluid impermeable membrane (or just “membrane”) can be interposed between an inlet port and an outlet port or at said outlet port of a chest tube. The membrane is configured to seal the tube's passageway from any transfer of the fluid. The impermeable membrane is configured to be ruptured by a force other than pressure from said fluid.

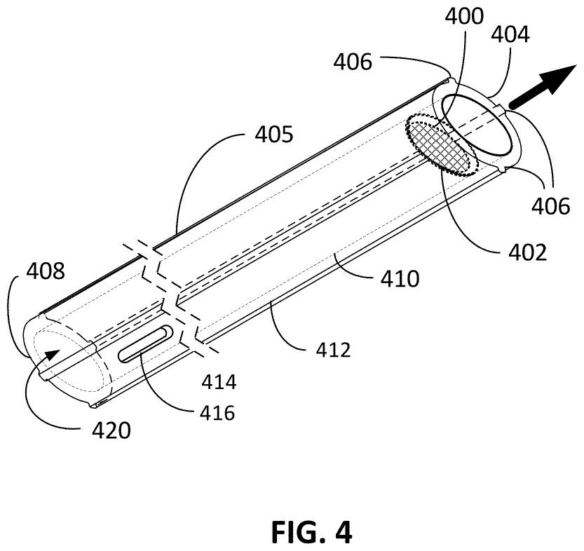

FIG. 4 illustratively depicts a perspective line drawing of a single use chest evacuation tube system embodiment consistent with embodiments of the present invention. As shown, a fluid impermeable membrane embodiment 400 (cross-hatched) is fixedly attached to the inner wall 410, perhaps by a collar or ring 402, to a single/unitary chest tube 405 between an inlet port 408 and an outlet port 404. Certain embodiments envision the impermeable membrane 400 disposed within two inches from the outlet port 404. The broken line 414 indicates that the chest tube 405 is much longer than that which is shown. The chest tube 405 is essentially a flexible hollow tube providing a passageway 4420 between the inlet port 408 and the outlet port 404. Chest cavity fluid and/or air moves through the chest tube 405 in the direction of the arrow when the chest tube is inserted, or deployed, in a chest cavity 288. In the present embodiment, the chest tube 405 comprises four ribbed members 406 that provide separation between the side inlet ports 416 and tissue within the chest cavity 288. That is, when the chest tube 405 is deployed in a subject 299, the ribbed members 406 create a tent-like space the keeps the chest cavity tissue from closing off the inlet ports 416. The net result is to improve the efficiency of body fluid escaping from the chest cavity 288. In the present embodiment, fluid and or air from the chest cavity 288 cannot travel through the passageway 420 and out from the outlet port 404 while the membrane 400 is intact.

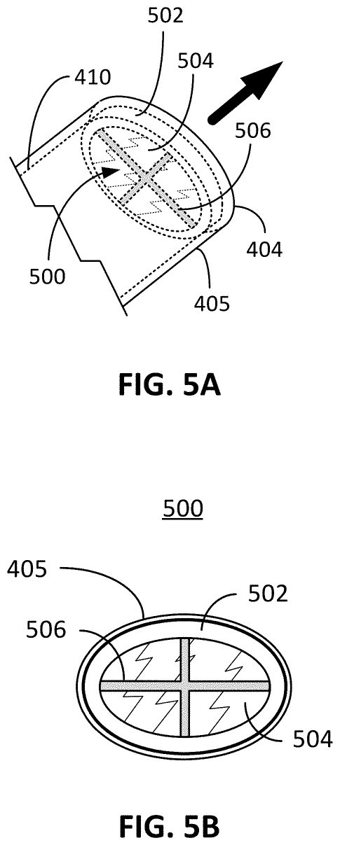

FIGS. 5A-5C illustratively depict drawings of a rupturable membrane embodiment consistent with embodiments of the present invention. The dashed lines show an element inside of another element, for example the membrane 500 disposed inside of the chest tube 405. FIG. 5A depicts a membrane embodiment 500 view from the bottom through the tube. The membrane 500 can generally be comprised of a collar 502 (which in some embodiments is a ring) with a membrane bladder 504 that is stretched across the inner circle defined by the ring 502, as shown. The bladder 504 can be a thin polymer material, such as Polyvinylidene Chloride (PVC) or Low-Density Polyethylene (LDPE), just to name several examples. Optional embodiments envision the bladder 504 composed of a metal foil, a wax paper, or other sheet material that is impermeable to body fluid and/or air. Hence, as long as the membrane is intact, body fluid and/or air is blocked, however when the membrane is ruptured, the body fluid/air will flow out of the chest tube 405 in the direction of the arrow. Here, the collar 502 is fixedly attached to the inner surface 408 of the chest tube 405, such as by adhesive or pressure fit, for example. Though the bladder 504 is depicted inside of the chest tube 405 here, the bladder 504 could just as easily be covering the outlet port 404 without departing from the scope and spirit of the present invention. Certain embodiments further envision the collar 502 conforming to the cross-sectional shape of the chest tube 405, which in this case is oval. In the present embodiment, the bladder 504 possesses failure lines 506, which are essentially stress concentration features in the membrane bladder 504. Accordingly, the bladder 504 is envisioned to fail, or otherwise burst, in a controlled manner along the failure lines 506.

FIG. 5B illustratively depicts a front view of the of the membrane 500 as viewed from the inlet port 404 of the chest tube 405. As is better appreciated from this view, the collar 502 is elliptically shaped to conform to an elliptically shaped chest tube 405. The membrane 500 is in a non-ruptured/pristine (new) condition that is effectively intact to function as an impermeable barrier to fluid and/or air, which effectively blocks the passageway 420 of the chest tube 405. The failure lines 506 in the bladder 504 as shown intact.

FIG. 5C illustratively depicts a front view of the membrane 500 whereby the bladder 504 is ruptured along the failure lines 506 from squeezing the chest tube 405 together (such as between an operator's fingers and thumb) while the membrane 500 is attached to the chest tube 405. In more detail, when intact, the membrane 500 is impermeably blocking the passageway 420 in the chest tube 405, however when the chest tube 405 is squeezed together as shown by the arrows 509, the bladder 504 ruptures 513 allowing fluid and/or air to pass. In this embodiment, the collar 502 complies with the chest tube 405 thus compressing together causing the bladder 504 to rupture 513 along the failure lines 506. Once ruptured, fluid and/or air is permitted to freely pass through the passageway 420 in the chest tube 405.

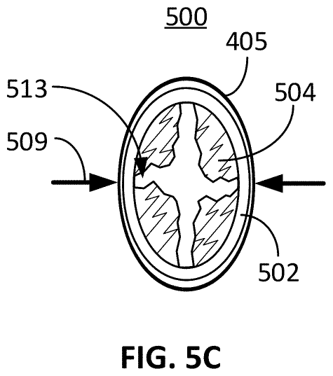

FIG. 6A-6C illustratively depicts line drawings of another membrane rupturing embodiment consistent with embodiments of the present invention. With reference to FIGS. 6A and 6B, the membrane 500 is ruptured by pressing a rigid or semirigid barbed tube 600 (or something more rigid than the pliable/flexible chest tube 405) through the collar 502 and bladder 504. The barbed tube 600 provides a hollow pathway 602 defined between the inlet aperture 604 and the outlet aperture 610. The barbed tube 600 comprises concentric barbed rings 606 and 608 that oppose one another. The concentric barbed rings 608 and 606 can form a pressure fit locking mechanism when pressed into a flexible tube, such as a flexible surgical tube and in this case the chest tube's outlet port 404. As shown in FIG. 6B, the bladder 504 is ruptured thereby creating an aperture/opening 615 leading through the hollow pathway 602 in the barbed tube 600. FIGS. 6A and 6B do not show the chest tube 405.

FIG. 6C illustratively depicts a line drawing of the barbed tube 600 pressed inside the outlet port 404 of the flexible chest tube 405. As shown, the barbed tube 600 deforms the softer (more pliable) chest tube 405 near the outlet port region 427 in order to form a pressure fit (i.e., fixedly attach to the tube from friction) inside of the chest tube 405. By forcing the barbed tube 600 into the outlet port 404, the barbed tube 600 also ruptures the bladder 504 of the membrane 500. Certain embodiments envision that the membrane 500 is disposed further from the outlet port 404 and towards the inlet port 408 but within range of where the barbed tube 600 can penetrate and rupture the membrane 500. The chest tube 405 extends much longer than is shown but for the sake of illustration is shortened at the break-line 622. Shown for reference is the outlet aperture 610.

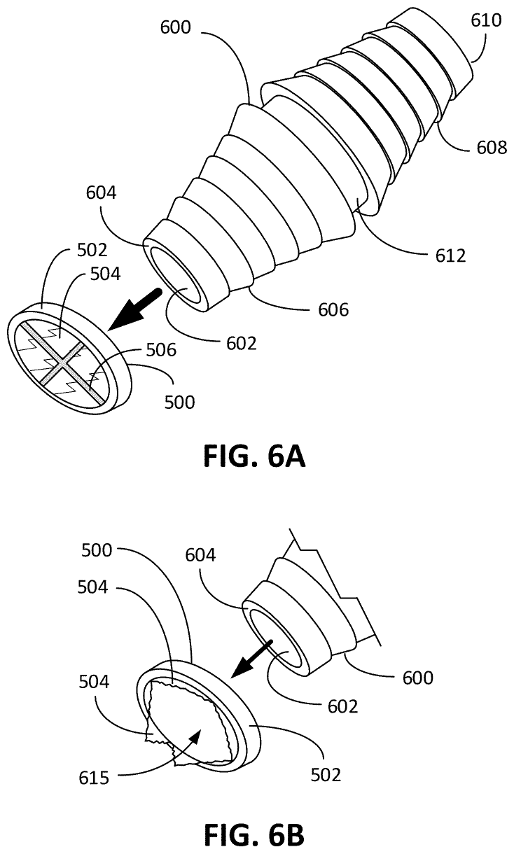

FIG. 7 illustratively depicts a line drawing of a hinged bladder system consistent with embodiments of the present invention. As shown here, the hinged bladder system 700 generally comprises a bladder 704 that can be rigid, semi-rigid, or flexible. The hinged bladder 704 can be attached to a collar 702 via hinge point 706. The hinge point 706 can be any number of mechanical hinges known to those skilled in the art. Considering the example wherein the bladder is a flexible material, the hinge point 706 can be any place where the bladder 704 is attached to the collar 702. Certain embodiments envision the bladder 704 being biased (via spring force) towards the interior aperture 708 of the collar 702 whereby without the benefit of a foreign object such as the barbed tube 600, the bladder 704 will swing shut mating with the collar lip 710 and closing off the interior aperture 708. When in a closed position (i.e., closing off the interior aperture 708), the bladder 704 covers or otherwise seals off the interior aperture 708. Though not shown, the collar 702 is adapted to be attached to the chest tube 405 in any number of ways, some of which are described above in conjunction with other collars/rings.

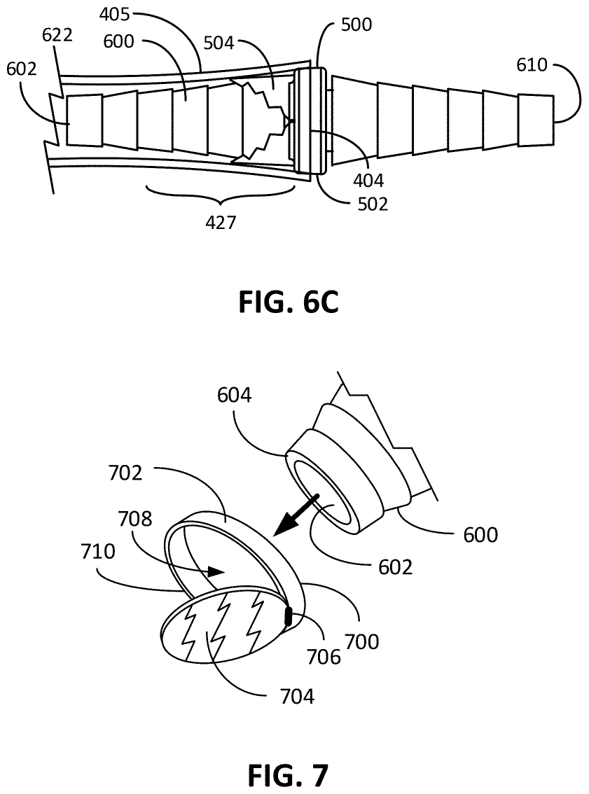

FIGS. 8A and 8B illustratively depict line drawings of yet another single use membrane-chest tube system consistent with embodiments of the present invention. As shown, the impermeable membrane 800 possesses a tab portion 802 extending from a bladder 804. The bladder 804 stretches across the outlet port 404 of the chest tube 405. More specifically, the bladder 804, considered the main body of the membrane 800, can be adhered to the lip, which defines the outlet port 404 of the chest tube 405 (the lip having a thickness defined at the distance between the outer wall and the inner wall 410 of the chest tube 405). Certain embodiments envision and adhesive bonding the bladder periphery to the chest tube outlet port lip 404 at a bonding interface 806 by way of an adhesive that is strong enough to prevent failure of the adhesive seal due to pressure from bodily fluid and/or air coming from a chest cavity 288. The adhesive is not strong enough to overcome the force generated when that tab 802 is manually pulled off of the chest tube outlet port lip 404. The tab 802 is configured to be pinched between an operator's thumb and finger (not shown). In operation, the chest tube 405 with the membrane 800 is inserted into a chest cavity 288 (via the inlet port 408), which has the effect of a sealed chest tube outlet port 401. Fluid and/or air can escape through the outlet port 404 only after the tab 802 is pulled in the direction of the arrow, which peels the membrane 800 from the bonding interface 806.

Certain embodiments envision a semipermeable membrane that slowly and in a controlled manner permits air and/or fluid to seep through the membrane, which also prevents splattering outside of the outlet port 404.

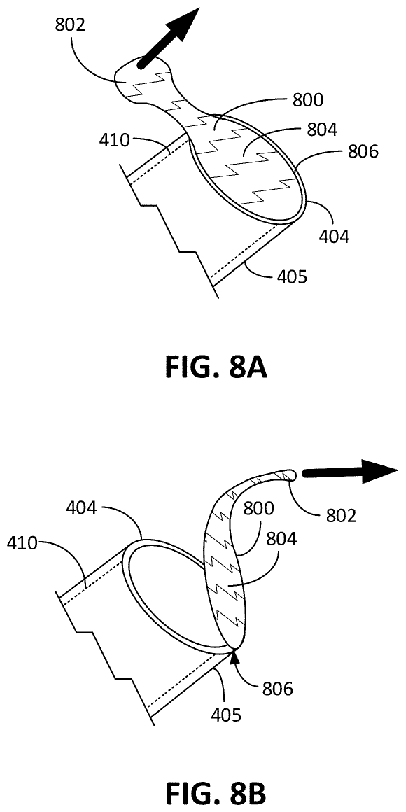

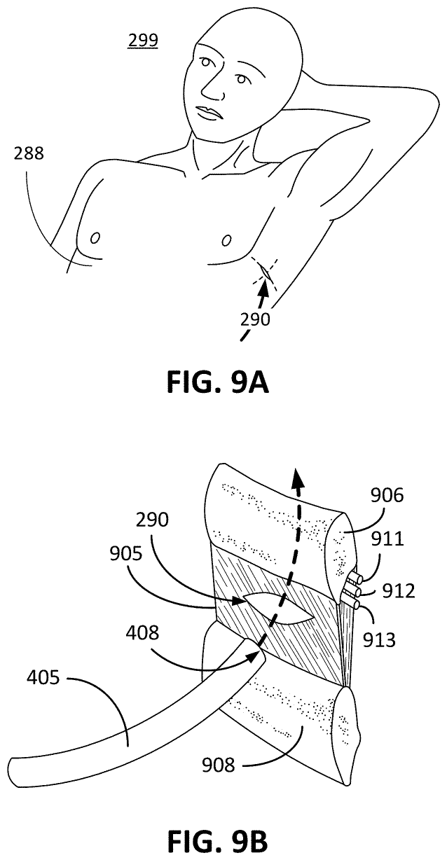

Certain embodiments of the present invention can be commercially used when managing a chest fluid evacuation procedure. FIG. 9A illustratively depicts a typical location where an embodiment of the integrated chest evacuation tube and membrane system can be deployed. As shown, the patient/subject 299 is marked with a dashed-X 290 pointing to a typical location at the fifth rib (under the armpit) where a membrane blocking chest evacuation tube can be deployed to alleviate fluid/air buildup possibly due to trauma, for example.

FIG. 9B illustratively depicts one embodiment of the chest evacuation tube 405 integrated with a membrane embodiment being inserted between ribs of a patient/subject consistent with embodiments of the present invention. Here, an incision 290 is made via the actuator scalpel 200 (not shown in this figure) in the intercostal muscles 805 between an upper rib 906 and a lower rib 908. Certain embodiments envision the integrated chest evacuation tube 405 and membrane system 500, 600, 700 or 800 or equivalent within the scope and spirit of the present invention being oval in cross-section of at least a curved polymer tube portion in order to fit more effectively between the ribs 906 and 908 while providing greater volume of liquid/air to drain out from buildup in the chest cavity 288. For reference, the intercostal artery 812 and the intercostal vein 811 are shown.

It is to be understood that even though numerous characteristics and advantages of various embodiments of the present invention have been set forth in the foregoing description, together with the details of the structure and function of various embodiments of the invention, this disclosure is illustrative only, and changes may be made in detail, especially in matters of structure and arrangement of parts within the principles of the present invention to the full extent indicated by the broad general meaning of the terms in which the appended claims are expressed. For example, though an integrated chest evacuation tube and membrane systems are depicted by example as a chest tube 405 and chest cannula 140, however, other embodiments could equally be used while still maintaining substantially the same functionality without departing from the scope and spirit of the present invention. Another example can include providing various other impermeable or low-permeable membrane systems that function in the same way directed to a chest evacuation tube without departing from the scope and spirit of the present invention. Though air and fluid are envisioned as two separate compositions that can escape through the tube or tunnel created by the integrated chest evacuation tube valve embodiments, from a physics point of view air is also considered a fluid, hence, if fluid is simply used to define compositions escaping through the integrated chest evacuation tube valve system, it is reasonable to consider that fluid includes air. Yet another example can include variations of a chest evacuation tube, such as using different kinds of structures in the chest evacuation tube including perforation holes, raised elements such as ribs, or other features apparent within the scope and spirit of the present invention. Further, the term “one” is synonymous with “a”, which may be a first of a plurality.

It will be clear that the present invention is well adapted to attain the ends and advantages mentioned as well as those inherent therein. While presently preferred embodiments have been described for purposes of this disclosure, numerous changes may be made which readily suggest themselves to those skilled in the art and which are encompassed in the spirit of the invention disclosed and as defined in the appended claims.

What is claimed is:

1. A single use chest evacuation tube consisting:
an inlet port adapted to intake fluid from a human chest cavity into said single use chest evacuation tube;
an outlet port adapted to expel said fluid out there through;
a single flexible hollow tube defining a passageway between said inlet port and said outlet port, said single flexible hollow tube adapted to transport said fluid from said input port to said output port; and
a fluid impermeable membrane 400 fixedly attached to said single flexible hollow tube, said fluid impermeable membrane 400 interposed between said inlet port 408 and said outlet port 404 or at said outlet port 404, said membrane 400 configured to seal said passageway 420 from said fluid exiting said outlet port 404, said impermeable membrane 400 configured to be ruptured by a force other than pressure from said fluid.

2. The single use chest evacuation tube of claim 1 wherein said impermeable membrane is ruptured when said single flexible hollow tube is squeezed where said fluid impermeable membrane is attached.

3. The single use chest evacuation tube of claim 2 wherein said impermeable membrane possesses at least one failure line along which said impermeable membrane is configured to fail when squeezed together.

4. The single use chest evacuation tube of claim 2 wherein said impermeable membrane comprises a collar that conforms to an inner cross-sectional shape of said passageway, and a ruptureable bladder that stretches to at least an inside periphery of said collar.

5. The single use chest evacuation tube of claim 4 wherein said collar having an outer edge that attaches to said passageway.

6. The single use chest activation tube of claim 4 wherein said ruptureable bladder covers said outlet port.

7. The single use chest activation tube of claim 1 wherein said impermeable membrane is adapted to be ruptured when a foreign object penetrates said impermeable membrane.

8. The single use chest activation tube of claim 7 wherein said foreign object is a rigid tube adapted to be pressed into said passageway via said outlet port and said rigid tube is adapted to be fixedly retained in said flexible hollow tube.

9. The single use chest activation tube of claim 8 wherein said impermeable membrane further comprises stress concentration lines, said impermeable membrane adapted to fail along said stress concentration lines when said rigid tube penetrates said impermeable membrane.

10. A closed system chest evacuation tube comprising:
an inlet port adapted to intake fluid from a human chest cavity into said single use chest evacuation tube;
an outlet port adapted to expel said fluid out of said single use chest evacuation tube through said outlet port;
one passageway between said inlet port and said outlet port, said passageway configured to transport said fluid from said input port to said output port; and
a membrane that is impermeable to said fluid, said membrane fixedly attached to said single use chest evacuation tube, said membrane fully blocks said passageway, said membrane configured to rupture by though physical manipulation other than pressure from said fluid.

11. The single use chest evacuation tube of claim 10 wherein said membrane comprises a collar that conforms to an inner cross-sectional shape of said passageway and a ruptureable bladder that stretches to at least an inside periphery of said collar.

12. The single use chest evacuation tube of claim 11 wherein said collar attaches within 2 inches of said outlet port.

13. The single use chest evacuation tube of claim 10 wherein said membrane comprises at least one region of failure.

14. The single use chest evacuation tube of claim 13 wherein said region of failure is a linear stripe in said membrane that is weaker than the rest of said membrane.

15. The single use chest evacuation tube of claim 13 wherein said membrane is adapted to fail when said single use chest evacuation tube is squeezed together.

16. The single use chest evacuation tube of claim 10 wherein said membrane is configured to be ruptured by way of a foreign object penetrating said membrane.

17. A method comprising:
providing a single use chest evacuation tube, an outlet port, a single passageway between said inlet port and said outlet port, a membrane fixedly attached to said single use chest evacuation tube, said membrane fully blocks said passageway;
pushing the inlet port into a human chest cavity under pressure with chest fluid;
after said pushing step, unblocking said passageway by rupturing said membrane through physical manipulation that does not include pressure from said chest fluid; and
after said rupturing step, directing said chest fluid that enters said inlet port, passes through said passageway, passes through said ruptured membrane and out though said outlet port.

18. The method of claim 17 wherein said membrane comprises a collar that conforms to a cross-sectional shape of said single use chest evacuation chest tube and a bladder that stretches to at least an inside periphery of said collar, said bladder comprising at least one weakened region that ruptures via said physical manipulation.

19. The method of claim 18 wherein said rupturing step is accomplished by squeezing said single use chest evacuation tube to compress said single passageway approximately where said membrane is attached.

20. The method of claim 17 wherein said rupturing step is accomplished by forcing a stiff hollow tube through said membrane.
